(19) United States
(12) Patent Application Publication
(10) Pub. No.: US 2026/0207677 A1
(43) Pub. Date: Jul. 23, 2026

(54) DERMAL REJUVENATION COMPOSITION COMPRISING PLACENTA DERIVED BIOMATERIAL AND USES THEREOF

(71) Applicant: Celularity Inc., Florham Park, NJ (US)

(72) Inventors: Anna GOSIEWSKA, Florham Park, NJ (US); Rajarajeswari SIVALENKA, Florham Park, NJ (US); Stephen A. BRIGIDO, Florham Park, NJ (US); Desiree LONG, Florham Park, NJ (US); Robert J. HARIRI, Florham Park, NJ (US)

(73) Assignee: Celularity Inc., Florham Park, NJ (US)

(21) Appl. No.: 19/145,935

(22) PCT Filed: Jan. 5, 2024
(86) PCT No.: PCT/US2024/010457

§ 371 Date: Jul. 5, 2025

Related U.S. Application Data
(60) Provisional application No. 63/478,559, filed on Jan. 5, 2023.

(51) Int. Cl. A61K 35/50; A61K 31/07; A61K 31/728; A61K 47/26; A61P 17/02

(52) U.S. Cl. CPC A61K 35/50; A61K 31/07; A61K 31/728; A61K 47/26; A61P 17/02

(57) ABSTRACT

Provided herein is a novel and useful dermal rejuvenation composition having applications in treating a skin disease or condition.

### PRIORITY CLAIM

This application claims priority to U.S. Provisional Application No. 63/478,559 filed 5 Jan. 2023, which is hereby incorporated by reference herein in its entirety.

### FIELD OF THE DISCLOSURE

The instant disclosure relates generally to the field of aesthetics and dermal treatments and provides a dermal rejuvenation composition and methods for treating skin diseases or conditions.

### BACKGROUND

Placental derived biomaterials, such as, e.g., placenta derived extracellular matrix, placenta derived cell conditioned media, placental derived exosomes, and placenta derived perfusate have been proposed to be immune-privileged such that, when presented physically and biochemically to a “native” administration site on a subject, they are proposed to rapidly re-establish healing cues and limit foreign body reactions.

Placenta acts as a supply of oxygen, detoxification, and hormone secretion in a pregnant mother, and its active ingredients include amino acids such as leucine, lysine and valine, and B1, B2, C, Vitamins such as D and E, minerals such as calcium, sodium and phosphorus, and many enzymes and nucleic acids and extracellular matrix proteins, such as collagen, elastin, laminin, fibronectin and sulphated glycosaminoglycans.

Japanese Patent Application Laid-Open No. 35-15399 discloses a color white cosmetic compound comprising placenta extract and koji acid or koji acid derivatives in a cosmetic base. The placenta extract and koji acid or koji acid derivative of this cosmetic compound provide the whitening effect by inhibiting the activity of tyrosinase by participating in the initial stage of melanogenesis. However, the use of this cosmetic compound has shown only limited success, if at all, in its ability to inhibit elastinase. Consequently, it has exhibited only a limited success in improving wrinkles.

Furthermore, Korean patent applications KR20040082144A, KR101414150B1, and KR20070117355A disclose, inter alia, skin topical placenta compositions containing placenta extract and methods for their preparation. These compositions comprise, inter alia, placenta extract prepared by the supercritical extraction method. Also disclosed are bioplacenta skin coating compositions that comprise placenta extract, organic germanium and ubiquinone (Coenzyme-Q10), which promote whitening of the skin and anti-aging of the skin. When applying these compositions to the skin, whitening, high melanin production suppression, moisturizing, anti-wrinkle and antioxidants, etc. were observed. In order to promote the survival, proliferation, and antibody-dependent cell cytotoxicity (ADCC) in the skin cells, IL-15 is co administered. This cytokine: (a) induces strong proliferative signals via JAK/STAT and Ras/MAPK signaling pathways, and (b) prevents cell death by increasing anti-apoptotic proteins Bcl-2 and Bcl-xL as well as decreasing pro-apoptotic proteins Bim and Puma through activation of PI3K pathway, and thus, supports cell expansion and maintenance (Huntington N D, Puthalakath H, Gunn P, Naik E, Michalak E M, Smyth M J, et al Interleukin 15-mediated survival of natural killer cells is determined by interactions among Bim, Noxa and Mcl-1. Nat Immunol. 2007; 8:856-63; Johnston J A, Bacon C M, Finbloom D S, Rees R C, Kaplan D, Shibuya K, et al. Tyrosine phosphorylation and activation of STAT5, STAT3, and Janus kinases by interleukins 2 and 15. Proc Natl Acad Sci USA. 1995; 92:8705-9; Miyazaki T, Kawahara A, Fujii H, Nakagawa Y, Minami Y, Liu Z J, et al. Functional activation of Jak1 and Jak3 by selective association with IL-2 receptor subunits. Science. 1994; 266:1045-7; Miyazaki T, Liu Z J, Kawahara A, Minami Y, Yamada K, Tsujimoto Y, et al. Three distinct IL-2 signaling pathways mediated by bcl-2, c-myc, and Ick cooperate in hematopoietic cell proliferation. Cell. 1995; 81:223-31; Adunyah S E, Wheeler B J, Cooper R S. Evidence for the involvement of LCK and MAP kinase (ERK-1) in the signal transduction mechanism of interleukin-15. Biochem Biophys Res Commun. 1997; 232:754-8; and Steelman L S, Pohnert S C, Shelton J G, Franklin R A, Bertrand F E, McCubrey J A. JAK/STAT, Raf/MEK/ERK, PI3K/Akt and BCR-ABL in cell cycle progression and leukemogenesis. Leukemia. 2004; 18:189-218). IL-15 also enhances the cytotoxic effector functions of lymphocytes by increasing production of a cytolytic pore forming protein, perforin, and death-inducing enzymes, granzymes A/B, through all three pathways (Imada K, Bloom E T, Nakajima H, Horvath-Arcidiacono J A, Udy G B, Davey H W, et al. Stat5b is essential for natural killer cell-mediated proliferation and cytolytic activity. J Exp Med. 1998; 188:2067-74; Teglund S, McKay C, Schuetz E, van Deursen J M, Stravopodis D, Wang D, et al. Stat5a and Stat5b proteins have essential and nonessential, or redundant, roles in cytokine responses. Cell. 1998; 93:841-50; and Farag S S, Caligiuri M A. Human natural killer cell development and biology. Blood Rev. 2006; 20:123-37).

What is needed though are compositions that rejuvenate skin and thus readily have applications in treating a dermal disease or condition, such as wrinkle lines, wrinkles, elastosis, extracellular matrix depletions, fibroblast depletions, photo damage, and scars, to name only a few.

The citation of any reference herein should not be deemed as an admission that such reference is available as prior art to the instant disclosure.

### SUMMARY OF THE DISCLOSURE

Broadly, the present disclosure extends to a dermal rejuvenation composition comprising:
(a) a placenta derived biomaterial;
(b) hyaluronic acid, an analog or derivative thereof, or a salt thereof;
(c) a vitamin;
(d) an amino acid or a peptide
(e) a microelement; and
(f) a lipid compound.

Various types of placenta derived biomaterial have applications in a dermal rejuvenation composition of the instant disclosure. Examples include, but certainly are not limited to a placenta derived extracellular matrix, a placenta derived cell conditioned media, placenta derived exosomes, placenta derived plasma or serum and placenta derived perfusate. Moreover, a dermal rejuvenation composition of the instant disclosure can include any combination of these placenta derived biomaterials.

As explained above, a dermal rejuvenation composition as disclosed herein comprises, inter alia, hyaluronic acid, an analog or derivative thereof, or a salt thereof. Numerous analogs or derivatives of hyaluronic acid, as well as numerous salts have applications in a dermal rejuvenation composition of the instant disclosures. Moreover, analogs, derivatives of salts of hyaluronic acid having applications herein can have varying molecular weights. Examples of derivatives and analogs include, but certainly are not limited to high molecular Hyaluronan (ORTHOVISC), a fluid mixture of hylan A and hylan B polymers (SYNVISC ONE and SYNVISC), highly purified, partially cross-linked sodium hyaluronate (NaHA) (MONOVISC AND GENVISC 850), hyaluronan (a linear polysaccharide with disaccharide repeats of d-glucuronic acid and N-acetyl-d-glucosamine), a high molecular weight viscoelastic hyaluronan (HYMOVIS), well as esters of hyaluronic acid, to name only a few. A particular hyaluronic acid, or analog or derivative or salt thereof having applications in a dermal rejuvenation composition of the instant disclosure is a highly purified high molecular weight hyaluronic acid, which as the property of retaining an amount of water 1,000 times its own molecular weight. Such a highly purified high molecular weight hyaluronic acid has sufficient hygroscopic properties to allow it to retain water at the surface of the skin where a dermal rejuvenation composition of the instant disclosure is administered. At such a point of administration, this highly purified, high molecular weight hyaluronic acid protects the skin from external aggressions that otherwise may alter its natural lipidic barrier and stops the peeling off of the horny layer of the skin. This type of hyaluronic acid provides a velvety skin feel because it retains water on the surface of the skin and blocks water loss from the skin. A particular example of such a hyaluronic acid is CRISTALHYAL, which is such a hyaluronic acid or salt thereof complexed with bentonite clay. Givaudan (Switzerland) markets CRISTALHYAL, in which this particular hyaluronic acid or salt thereof is complexed with bentonite clay. The hyaluronic acid contained in CRISTALHYAL is identical to endogenously produced hyaluronic acid made from fibroblasts and keratinocytes. CRISTALHYAL is a white, water-soluble powder, preservation free, and the dosage at which it is administered to a subject range from about 0.01 to about 0.3% (w/w?w/v?). The molecular weight of hyaluronic acid contained in CRISTALHYAL ranges from about 700 kDa to about 2200 kDa. Naturally, medium molecule weight hyaluronic acid also has applications in a dermal composition of the instant disclosure, such as, for example having a molecular weight ranging from 100 kDa to about 300 KDa), such as, e.g. PRIMALHYAL 300, also produced by Givaudan.

Furthermore, numerous types of vitamins have applications in a dermal rejuvenation composition of the instant disclosure, such as, for example, retinol (vitamin A), ascorbic acid as well as analogs and derivatives thereof, e.g., ascorbyl palmitate (vitamin C), sodium riboflavin phosphate, thiamine HCl, vitamin B3 (niacin or an niacin amide (vitamin B3), pantothenic acid (vitamin B5), pyridoxine (vitamin B6), biotin (vitamin H), phylloquinone (vitamin K1), menaquinone (vitamin K2) calciferol (vitamin D), alpha-linolenic acid (ALA) and linoleic acid (LA) (vitamin F), coenzyme Q10 (vitamin Q10), and Cyanocobalamin (vitamin B12), to name only a few. Naturally, any combination of these vitamins also readily has applications in a composition of the instant disclosure.

Likewise, numerous amino acid acids, as well as salts thereof, and analogs or derivatives thereof. Particular examples of amino acids having applications herein comprise (a) Arg, (b) Asp, (c) Ala, (d) Ser, (e) Val, (f) Pro, (g) Thr, (h) Ile, (i) His, and (j) Phe. Naturally, any combination of these amino acids or salts or analogs or derivatives thereof readily have applications in a dermal rejuvenation composition of the instant disclosure. As explained above, peptides also have applications in a dermal rejuvenation composition of the instant disclosure.

Moreover, numerous lipid compounds readily have applications in a dermal rejuvenation composition as disclosed here, Examples include, but certainly are not limited to glycerin, nourishing oils or any combination thereof. Particular examples of nourishing oils having applications herein include, but certainly are not limited to jojaba oil, rosehip seed oil, argan oil and olive oil.

Moreover, numerous types of carbohydrates have applications in a dermal rejuvenation composition as disclosed herein. Examples include monosaccharides, e.g., glucose, fructose, etc., inositol, and glycerin, to name only a few.

9. The dermal rejuvenation composition of any of Claims 1-8, further comprising retinol or a derivative thereof.

In addition, the present disclosure extends to a dermal rejuvenation composition as disclosed herein, further comprising retinal or a derivative thereof, such as sodium retinoyl hyaluronate. A combination of retinal and an analog or derivative thereof also has applications in a dermal rejuvenation composition of the instant disclosure.

A dermal rejuvenation composition can be in many forms for administration, include but not limited to a fluid (such as a liquid (e.g. aqueous)), a solid, a solid processed into microparticles that are administered, or alternatively the microparticles are contained within a biodegradable or bioerodable polymer such as, for example, PLGA, PLA, a hydrogel, etc., which is then administered to a subject. In a particular embodiment, an aqueous dermal rejuvenation composition of the instant disclosure can have a pH that varies. For example, in one embodiment, the pH is about 5.0 to about 6.5. In another embodiment, an aqueous dermal rejuvenation composition of the instant disclosure has a pH of about 5.5 to about 6.0.

Furthermore, a personalized dermal rejuvenation composition of the instant disclosure can be personalized and based upon the specific needs of a subject. Such a personalized dermal rejuvenation composition of the instant disclosure can comprise highly tailored composition of ingredients for specific skin conditions, including, but not limited to skin elastosis, hyperpigmentation, skin aging, dark circles, eye puffiness, hair follicle regeneration. scar or any combination of above, and such a composition of hyaluronic acid, HYRETIN, an ester of hyaluronic and retinoic acid, vitamins, growth factors, peptides, amino acids complex, such as Prodew®500, antioxidants, micronutrients, anti-inflammatory components, such as extract from Centella asiatica, a herbaceous plant belonging to the Apiaceae (Umbelliferae) family. Additional anti-inflammatory and anti-inflammatory agents include glycyrrhizic acid and its derivatives, glycyrrhetinic acid derivatives, salicylic acid derivatives, hinokitiol, guaiazulene, allantoin, indomethacin, zinc oxide, hydrocortisone acetate, prednisone, diphedramine hydrochloride, chlorpheniramine maleate; peach leaf extract A plant extract such as a koji leaf extract are readily have applications in a dermal rejuvenation composition of the instant disclosure.

Furthermore, the instant disclosure extends to a method for treating a dermal disease or condition in a subject, comprising administering to the subject a therapeutically effective amount of a dermal rejuvenation composition as disclosed herein. Numerous dermal diseases or conditions can be treated with a method of the instant disclosure, such as wrinkle lines, wrinkles, elastosis, extracellular matrix depletion fibroblast depletions, photo damage, a scar, as well as any combination of these dermal diseases or conditions. A scar that can be treated with a method of the instant disclosure includes an acne scar, a burn scar, or a laceration scar.

Numerous routes of administration of a therapeutically effective amount of a dermal rejuvenation composition as disclosed herein have applications in a method of the instant disclosure. Such routes include topical administration, intradermal administration, or subcutaneous administration.

The present disclosure further extends to the use of a dermal rejuvenation composition as disclosed herein for use in a method for treating a dermal disease or condition in a subject, such as wrinkle lines, wrinkles, elastosis, fibroblast depletions, photo damage, a scar, or any combination thereof; and (g) any combination of (a)-(f).

Moreover, disclosed herein is a kit for treating a dermal disease or condition, comprising a dermal rejuvenation composition of the instant disclosure, a delivery device, and directions for use of the kit. In such a kit, the dermal rejuvenation composition can be in many forms for administration, include but not limited to a fluid (such a liquid (e.g. aqueous) or a salve, a solid, a solid processed in microparticles that are administered, or alternatively are contained within a microparticles of a biodegradable or erodible polymer such as, for example, PLGA, PLA, a hydrogel, etc., which is administered to a subject. If in a kit of the instant disclosure, the dermal rejuvenation composition is in aqueous form, it has a pH is about 5.0 to about 6.5, or about 5.5 to about 6.0. The delivery device contained in the kit can vary, depending upon the physical state of the composition and the point of administration. For example, for topical administration, the kit can include the composition contained within a microneedling device, a roller, or a pen to create micro-injuries in the skin, which allow for better absorption of a dermal rejuvenation compound of the instant disclosure. If intended for administration beneath the surface of the skin, the kit can include a microinjecting device such as, for example, an AQUAGOLD FINE TOUCH microinjection device Aquavit Pharmaceuticals), with needle depth of about 0.6 mm, twenty microchannels that contain miniature spiral groves constructed to microdose and microinject therapeutics. Such a device delivers about 1,200 to about 2,000 microinjections permit. Another Devices also having applications in a kit of the instant disclosure are a hydra needle 20 microneedle device with depth of about 0.25 or about 0.5 mm, with 64 pin needles made of titanium. Another example of a microneedling device having applications in a kit of the instant disclosure is the DERMAPEN microneedling device, which is a handheld microneedling device that is designed for professional and medical-grade skin treatments. The DERMAPEN device typically has adjustable needle depth settings, allowing for customization based on the specific treatment area and skin concerns. Alternatively, there are at-home microneedling devices that can be utilized that have shorter needles and may not be as powerful as those used in professional settings, such as, for example, the GLOPRO Microneedling Regeneration Tool. This microneedling tool is a handheld microneedling device designed for personal use, allowing individuals to perform microneedling treatments. Other devices, such as a syringe or a pen device have applications in a kit of the instant disclosure. The pen device can be refillable or disposable. Examples of such pen devices having applications in a kit of the instant disclosure include, but certainly not limited to, Sanofi's SOLARSTAR disposable pen, Novo Nordisk's FLEXPEN or NOVOPEN devices, or various pen devices produced by IPSOMED (Switzerland). If the composition is in the form of microparticles of a biodegradable or bioerodible polymer, an implant applicator, such as those, for example, disclosed in U.S. Pat. No. 10,849,656, which is hereby incorporated by reference in its entirety, can be included in a kit of the instant disclosure.

Furthermore, as part of delivery, a dermal rejuvenation composition of the instant disclosure can be combined with procedures like Morpheus8 (microneedling with radiofrequency) or laser resurfacing or any other dermatological procedure to enhance the overall results by providing additional nourishment, hydration, and support for the skin or hair recovery.

These and other aspects of the present disclosure will be better appreciated by reference to the following drawings and Detailed Description.

### BRIEF DESCRIPTION OF THE DRAWINGS

FIG. 1 is a photograph of the hands of a subject to whom a dermal rejuvenation composition of the instant disclosure in aqueous form was topically administered with placenta derived extracellular matrix to the left hand of a subject, and a saline solution was topically administered to the subject's right hand. The photo was taken after administration of the composition and the saline solution.

FIG. 2 is a photograph of the hands of a subject to whom a dermal rejuvenation composition of the instant disclosure in aqueous form was topically administered with placenta derived extracellular matrix to the left hand of a subject, and saline solution was topically administered to the subject's right hand. The photograph was taken five minutes after administration of the dermal rejuvenation composition and saline solution.

FIG. 3 is a photograph of a subject's hands following administration of a composition of the instant disclosure with placenta derived extracellular matrix. The left hand was treated with a dermal rejuvenation composition of the instant disclosure and the right hand was treated with saline (control). After five minutes exposure, the treatments were gently wiped off. The treated left-hand skin surface showed the appearance of a smoother and plumper skin when compared to the right-hand skin surface, which had an unchanged appearance.

### DETAILED DESCRIPTION

Broadly, the present disclosure extends to a dermal rejuvenation composition comprising:
(a) a placenta derived biomaterial;
(b) hyaluronic acid, an analog or derivative thereof, or a salt thereof;
(c) a vitamin;
(d) an amino acid or peptide;
(e) a microelement; and
(f) a lipid compound.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure.

All numerical designations, e.g. volume, mass, number of resin particles, etc. are approximations which are varied by (+) or (−) by increments of 1.0 or 0.1, as appropriate. It is to be understood, although not always explicitly stated, that all numerical designations are preceded by the term “about”.

Numerous terms and phrases are used throughout the instant specification and claims and are defined below.

“About” and “approximately” are interchangeable and mean plus or minus a percent (e.g. ±5%) of the number, parameter, or characteristic so qualified, which would be understood as appropriate by a skilled artisan to the scientific context in which the term is utilized.

As used herein, the singular form “a”, “an” and “the” include plural reference unless the context clearly dictates otherwise.

As used herein, the terms “comprising”, “comprises” and “comprise” are intended to mean that the compositions, preparations and methods disclosed herein include recited elements, but do not exclude others.

Therefore, if appearing herein, the following terms and phrases shall have the definitions set out below.

As used herein the term “placenta-derived” with respect to a biomaterial indicates that the source of the biomaterial is the placenta, which is typically obtained from animals (such as pigs or sheep) or, from human placental tissue.

As used herein, the phrase “placenta-derived cell-conditioned media (PCCM)” refers to a substance derived from the culture of cells obtained from the placenta. The placenta is a temporary organ that develops during pregnancy, and it plays a crucial role in providing nutrients and oxygen to the developing fetus. In the context of skincare or regenerative medicine, placenta-derived products, such as cell-conditioned media, are used for their potential benefits.

As used herein, the phrase “placenta derived cell-conditioned media (CCM)” refers to the liquid or media in which cells have been cultured. Cells secrete various bioactive molecules, including growth factors, cytokines, and other signaling molecules, into the culture media. When the media containing these secreted factors are collected, it is termed “conditioned media”. The collected media contains a complex mixture of proteins, growth factors, and other signaling molecules that were produced and secreted by the placental cells during the culture period. In the context of skincare or regenerative medicine, placenta-derived cell-conditioned media have potential benefits for skin health and rejuvenation. It is claimed that the growth factors and other bioactive molecules present in the conditioned media may contribute to promoting cell regeneration, collagen production, and overall skin vitality.

As used herein, the phrase ‘nourishing oils” refers to oils that are included in a cosmetic or skincare formulation with the purpose of providing essential nutrients and moisture to the skin. These oils are chosen for their potential benefits in promoting skin health, hydration, and overall rejuvenation. The term “nourishing” implies providing essential nutrients, vitamins, and minerals to support the health and well-being of the skin. Nourishing oils are selected for their ability to replenish and revitalize the skin.

“oils” are lipid-based substances derived from various sources, including plants, seeds, fruits, and nuts. These oils can be rich in fatty acids, antioxidants, and other beneficial compounds. Essential fatty acids found in oils, such as omega-3 and omega-6, can help maintain the skin's barrier function and prevent moisture loss. Furthermore, nurturing oils contain antioxidants, such as vitamin E and polyphenols, which help protect the skin from oxidative stress and environmental damage.

One of the properties of a moisturizing oil is the ability to moisturize, which helps hydrate the skin and improve its texture. In a skin rejuvenation composition of the instant disclosure, nourishing oils may be combined with other active ingredients, such as peptides, vitamins, and botanical extracts, to create a comprehensive formulation that addresses various aspects of skin health and rejuvenation. The goal is often to provide a synergistic effect, where the combined ingredients work together to enhance the overall efficacy of the product. Examples of nourishing oils having applications in a dermal rejuvenation composition of the instant disclosure include Jojoba Oil, Known for its moisturizing properties and similarity to the skin's natural oils; rosehip Seed Oil, which is rich in antioxidants and fatty acids, which are believed to promote skin regeneration; argan Oil, which contains vitamin E and fatty acids, which contribute to skin hydration, and olive, which possess moisturizing and antioxidant properties.

As used herein, the term “wrinkle” or “wrinkles” as used herein, refers to creases, folds, or ridges in the skin that develop over time as a natural part of the aging process. Wrinkles can be caused by a combination of factors, including aging, sun exposure, smoking, genetics, and repetitive facial expressions. Wrinkles can be categorized into different types, such as fine lines, deep furrows, and dynamic wrinkles (formed by repeated muscle movements).

As used herein, the phrase “wrinkle lines” refers to lines or creases on the skin's surface that result from the aging process. Similar to wrinkles, the development of wrinkle lines is influenced by aging, sun damage, and other environmental factors. The term “wrinkle lines” may imply a finer or more superficial type of wrinkle. It can refer to the early, subtle signs of aging, such as fine lines that appear on the skin's surface before deeper wrinkles develop. While both terms generally refer to the visible signs of aging on the skin, “wrinkles” is a broader term that encompasses various types of skin creases, including fine lines and deep furrows. “Wrinkle lines” describe the more superficial or finer lines that are often among the early signs of aging.

As used herein, the term “vitamin” refers to an organic compound that is essential for normal growth and nutrition and is required in small quantities in the diet because the body cannot synthesize it. The identification of each vitamin's common name is set forth in Table 1 below:

TABLE 1

| Vitamin | Common Name(s) and |
|---|---|
| Vitamin A | Retinol, Retinal, Retinyl Esters, Retinoic Acid |
| Vitamin B1 | Thiamine, Thiamine HCl |
| Vitamin B2 | Riboflavin. Sodium Riboblavinphosphate |
| Vitamin B3 | Niacin |
| Vitamin B5 | Pantothenic Acid |
| Vitamin B6 | Pyridoxine, Pyridoxal, Pyridoxamine |
| Vitamin B12 | Cobalamin |
| Vitamin H | Biotin |
| Vitamin C | Ascorbic Acid, Ascorbyl Palmitate |
| | choline |
| Vitamin D | Calciferol |
| Vitamin E | Tocopherol |
| Vitamin B9 | Folic Acid, Folate, Folacin |
| Vitamin K1 | Phylloquinone |
| Vitamin K2 | Menaquinone |
| Vitamin F | Alpha-Linolenic Acid, Linolenic Acid |
| Vitamin Q10 | Coenzyme Q10 |

As explained above, numerous types of vitamins, as well as analogs, derivatives or salts thereof, have applications in a dermal rejuvenation composition of the instant disclosure, such as, for example, retinol (vitamin A), ascorbic acid as well as analogs and derivatives thereof, e.g., ascorbyl palmitate, ascorbic acid fatty acid esters such as ascorbyl tetraisopalmitate, ascorbyl stearate, ascorbyl palmitate and ascorbyl dipalmitate, ascorbic acid alkyl ethers such as ascorbic acid ethyl ether, ascorbic acid glucosides such as ascorbic acid-2-glucoside and ascorbic acid derivatives such as fatty acid esters and tocopheryl phosphate ascorbyl; retinol acetate, and retinol palmitate (vitamin C), sodium riboflavin phosphate (vitamin B1, thiamine HCl, (vitamin B2, niacin or an niacin amide (vitamin B3), pantothenic acid (vitamin B5), pyridoxine (vitamin B6), biotin (vitamin H), phylloquinone (vitamin K1), menaquinone (vitamin K2) calciferol (vitamin D), alpha-linolenic acid (ALA) and linoleic acid (LA) (vitamin F), y, and 5-tocopherol as well as derivatives such as tocopherol nicotinate, tocopherol acetate, tocopherol acetate, tocopherol linoleate, tocopherol phosphate, tocopherol phosphate etc., tocotrienol (vitamin E), coenzyme Q10 (vitamin Q10), and Cyanocobalamin (vitamin B12), to name only a few.

As used herein, the term “microelement” refers to a trace element or a micronutrient that plays a crucial role in supporting various biochemical processes in the body. These elements are typically required in smaller amounts compared to macronutrients like carbohydrates, proteins, and fats. Micronutrients of a rejuvenation formulation include for example Zinc, an essential trace element that serves as a cofactor for numerous enzymes involved in various physiological processes. It plays a key role in supporting immune function, DNA synthesis, cell division, and wound healing; Zinc is known for its antioxidant properties, which can help combat oxidative stress and contribute to skin health. Additionally, it plays a role in collagen synthesis, promoting the maintenance and repair of connective tissues. Other examples not limited to the following include Vitamin C (Ascorbic Acid), an antioxidant that supports collagen synthesis, wound healing, and helps protect cells from oxidative stress. It promotes skin health, reduces the appearance of aging signs, and supports overall tissue repair. Vitamin E (Tocopherol): Antioxidant that protects cell membranes from damage and supports skin health. Helps combat oxidative stress, contributing to a more youthful appearance. Selenium acts as a cofactor for antioxidant enzymes, helps regulate thyroid function that supports antioxidant defense mechanisms and may contribute to skin health. Another example is copper, which is a cofactor for enzymes involved in collagen synthesis and antioxidant defense. It contributes to the maintenance of connective tissues, promoting skin elasticity.

As used herein, the phrase “extracellular matrix” refers to a collagen-rich substance that is found in between cells in mammalian tissue, and any material processed therefrom, e.g. decellularized ECM. Naturally, placenta derived extracellular matrix is derived from the placenta. Numerous types of extracellular matrices have applications in a dermal rejuvenation composition of the instant invention. Examples include, but certainly are not limited to those disclosed in issued U.S. Pat. No. 7,928,280 and US patent publication US20130184821, each of which is hereby incorporated by reference herein in their entireties.

As used herein, the terms “exosome” or “exosomes” refer to naturally existing nanoparticles that are secreted endogenously by many types of in vitro cell cultures and in vivo cells, and are commonly found in vivo in body fluids, such as blood, urine and malignant ascites. Exosomes are cup-like multivesicular bodies (MVBs) varying in size between 30-100 nm. MVBs are specialized endosomes in the endocytosis pathway of cells and are formed by inward budding and scission of vesicles from the limiting membranes into the endosomal lumen. During the formation of MVBs, transmembrane and peripheral membrane proteins are absorbed into the vesicle membrane, and at the same time, cytosolic components are also embedded in the vesicles. As this process progresses, the MVBs ultimately fuse with the cellular membrane, triggering the release of the exosomes from the cells.

As used herein, the phrase “therapeutically effective amount” refers to As used herein the phrase “effective amount” refers to an amount of a therapeutic composition (e.g., a dermal rejuvenation composition of the instant disclosure) sufficient to produce a measurable biological response. Actual dosage levels of active ingredients in a dermal rejuvenation composition of the instant disclosure can be varied so as to administer an amount that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

As used herein, the phrase “amino acid” refers to organic compounds that contain both amino and carboxylic acid functional groups. Twenty amino acids are considered the building blocks of proteins. These are:
Phenylalanine (Phe or F)
Leucine (Leu or L)
Isoleucine (He or I)
Methionine (Met or M)
Valine (Val or V)
Serine (Ser or S)
Proline (Pro or P)
Threonine (Thr or T)
Alanine (Ala or A)
Tyrosine (Tyr or Y)
Histidine (His or H)
Glutamine (Gln or Q)
Asparagine (Asn or N)
Lysine (Lys or K)
Aspartic Acid (Asp or D)
Glutamic Acid (Glu or E)
Cysteine (Cys or C)
Arginine (Arg or R)
Glycine (Gly or G)
Tryptophan (Trp or W)

A number of these amino acids readily have applications in a dermal rejuvenation composition of the instant disclosure, such as (a) Arg, (b) Asp, (c) Ala, (d) Ser, (e) Val, (f) Pro, (g) Thr, (h) Ile, (i) His, and (j) Phe. Naturally, any combination of these amino acids or salts or analogs or derivatives thereof readily have applications in a dermal rejuvenation composition of the instant disclosure.

Examples of chelating agents having applications in a dermal rejuvenation composition of the instant disclosure include, but certainly are not limited to edetate (ethylenediaminetetraacetate) such as EDTA, EDTA2Na, EDTA3Na, and EDTA4Na; hydroxyethylethylenediaminetriacetate such as HEDTA3Na; pentetate (diethylenetriaminepentaacetate); phytic acid; Phosphonic acid and its sodium salt; sodium oxalate; polyamino acids such as polyaspartic acid and polyglutamic acid; sodium polyphosphate, sodium metaphosphate, phosphoric acid; sodium citrate, citric acid, alanine, dihydroxyethylglycine Gluconic acid, ascorbic acid, succinic acid, and tartaric acid.

As explained above, a dermal rejuvenation composition of the instant disclosure has a pH of about 5.0 to about 6.5, and in particular, of about 5.5 to about 6.0. Numerous compounds can be used to adjust the pH to the appropriate level. For example, pH adjusters/acids/alkalis can be used to adjust the pH. Examples include, but certainly are not limited to citric acid, sodium citrate, lactic acid, sodium lactate, glycolic acid, succinic acid, acetic acid, sodium acetate, malic acid, tartaric acid, fumaric acid, phosphoric acid, hydrochloric acid, sulfuric acid, monoethanol Amine, diethanolamine, triethanolamine, isopropanolamine, triisopropanolamine, 2-amino-2-methyl-1,3-propanediol, 2-amino-2-hydroxymethyl-1,3-propanediol, arginine, hydroxylated Sodium, potassium hydroxide, aqueous ammonia, guanidine carbonate, and ammonium carbonate. In a particular embodiment, ammonium carbonate is used to adjust the pH.

### Kits

Provided herein is a pack or kit for treating a dermal disease or condition, comprising a dermal rejuvenation composition of the instant invention, a delivery device, and directions for use of the kit. In such a kit, the dermal rejuvenation composition can be in many forms for administration, include but not limited to a fluid (such a liquid (e.g. aqueous) or a salve, a solid, a solid processed in microparticles that are administered, or alternatively are contained within a microparticles of a biodegradable or bioerodible polymer such as, for example, PLGA, PLA, a hydrogel, etc., which is administered to a subject. If in a kit of the instant disclosure, the dermal rejuvenation composition is in aqueous form, it has a pH is about 5.0 to about 6.5, or about 5.5 to about 6.0. The delivery device contained in the kit can vary, depending upon the physical state of the composition and the point of administration. For example, for topical administration, the kit can include the composition contained within a microneedling device, a roller, or a pen to create micro-injuries in the skin, which allow for better absorption of a dermal rejuvenation compound of the instant disclosure. If intended for administration beneath the surface of the skin, the kit can include a microinjecting device such as, for example, an AQUAGOLD FINE TOUCH microinjection device Aquavit Pharmaceuticals), with needle depth of about 0.6 mm, twenty microchannels that contain miniature spiral groves constructed to microdose and microinject therapeutics. Such a device delivers about 1,200 to about 2,000 microinjections permit. Another Devices also having applications in a kit of the instant disclosure are a hydra needle 20 microneedle device with depth of about 0.25 or about 0.5 mm, with 64 pin needles made of titanium. Another example of a microneedling device having applications in a kit of the instant disclosure is the DERMAPEN microneedling device, which is a handheld microneedling device that is designed for professional and medical-grade skin treatments. The DERMAPEN device typically has adjustable needle depth settings, allowing for customization based on the specific treatment area and skin concerns. Alternatively, there are at-home microneedling devices that can be utilized that have shorter needles and may not be as powerful as those used in professional settings, such as, for example, the GLOPRO Microneedling Regeneration Tool. This microneedling tool is a handheld microneedling device designed for personal use, allowing individuals to perform microneedling treatments. Other devices, such as a syringe or a pen device have applications in a kit of the instant disclosure. The pen device can be refillable or disposable. Examples of such pen devices having applications in a kit of the instant disclosure include, but certainly not limited to, Sanofi's SOLARSTAR disposable pen, Novo Nordisk's FLEXPEN or NOVOPEN devices, or various pen devices produced by IPSOMED (Switzerland). If the composition is in the form of microparticles of a biodegradable or bioerodible polymer, an implant applicator, such as those, for example, disclosed in U.S. Pat. No. 10,849,656, which is hereby incorporated by reference in its entirety, can be contained in a kit of the instant disclosure. The kit of the instant disclosure can be used in accordance with a method described herein, e.g., a method of treating a skin disease or condition, or with a use described herein of a dermal rejuvenation composition of the instant disclosure.

The present disclosure may be better understood by reference to the following non-limiting examples, which are provided as exemplary of the disclosure. The following examples are presented in order to more fully illustrate the preferred embodiments of the disclosure. They should in no way be construed, however, as limiting the broad scope of the disclosure.

### EXAMPLES

The instant disclosure provides a dermal rejuvenation composition and methods for treating the skin diseases or conditions. The compositions containing base skin micronutrients including, but not limited to sodium hyaluronate, vitamins, amino acids, microelements and lipids and at least one additional constituent selected from the group consisting of placental derived extracellular matrix, placental derived cell conditioned media, placental exosomes, placental perfusate, wherein the composition with additional constituents delivered via microinfusion. Methods for treating lines, wrinkles, elastosis, fibroblast depletions, photodamage, acne scars, hair restoration with the disclosed compositions are provided as well.

### Introduction

Placental extracellular matrix (ECM) based biomaterials have been proposed to be immune-privileged that when presented physically and biochemically “native” matrices to implants sites, placental ECM biomaterials are proposed to rapidly re-establish healing cues and limit foreign body reactions.

Placenta acts as a supply of oxygen, detoxification, and hormone secretion in a pregnant mother, and its active ingredients include amino acids such as leucine, lysine and valine, and B1, B2, C, Vitamins such as D and E, minerals such as calcium, sodium and phosphorus, and many enzymes and nucleic acids and extracellular matrix proteins, such as collagen, elastin, laminin, fibronectin and sulphated glycosaminoglycans.

Review of a prior art described the use of the placenta extract, by Japanese Patent Application Laid-Open No. 35-15399 which discloses a color white cosmetic compound comprising placenta extract and koji acid or koji acid derivatives in a cosmetic base. The placenta extract and koji acid or koji acid derivative of the above cosmetics provide the whitening effect by inhibiting the activity of tyrosinase by participating in the initial stage of melanogenesis, but there is a problem that the wrinkle improvement effect by the inhibition of elastinase is weak.

In another disclosures, KR20040082144A, KR101414150B1, KR20070117355A, South Korea, skin topical bioplacenta composition containing placenta extract and method for preparation are described, in particular a placenta extract prepared by the supercritical extraction method and a bioplacenta skin coating composition comprising organic germanium and ubiquinone, that have a function of whitening and anti-aging, and include placenta extract, organic germanium and ubiquinone (Coenzyme-Q10). When applying the skin coating composition to the skin, it was reported that the effect of whitening, high melanin production suppression, moisturizing, anti-wrinkle and antioxidant, etc., as the basis of the invention.

Provided herein is a water-limited base composition, using selected ingredients, including, but not limited to sodium hyaluronate, nourishing oils, hyaluronic acid, vitamins, amino acids, peptides creating more potent composition while diminishing the need for preservatives and unnecessary fillers that allergize skin. This base composition is formulated with various compositions of sodium hyaluronate of different molecular weights to enhance its penetration to the skin, vitamins, peptides, amino acids, and retinol, delivered via micro-infusion. The base composition can be supplemented with at least one additional constituent selected from the group consisting of placental derived extracellular matrix, placental derived cell conditioned media, placental exosomes, placental perfusate, wherein the composition with additional constituents is delivered via microinfusion. The additional constituents selected from the group consisting of placental-derived ingredients, placental adherent mesenchymal-like cells, (PDAC) conditioned media, PDAC-derived micronutrients, placental exosomes, placental perfusate and placental extracellular matrix (ECM) to provide personalized solutions for different skin conditions, including skin elastosis, hyperpigmentation, skin aging, hair maintenance or treatment. The dermal rejuvenation composition of the instant disclosure proactively targets hard-to-address, specific skin needs by filling the dermis of the skin.

The instant disclosure relates generally to the field of aesthetics and dermal treatments including compositions composed of placental derived ingredients such as supernatants from Placental derived adherent cell culture, Extracellular Matrix (ECM), Exosomes, umbilical cord plasma or serum, Placental Perfusate Serum, in compositions to be delivered by methods including micro infusion, micro needling, rolling. The invention provides dermal rejuvenation composition and methods for treating the appearance of the skin. The compositions containing base skin micronutrients including, but not limited to sodium hyaluronate, Sodium retinoyl hyaluronate, vitamins, amino acids, microelements and lipids and at least one additional constituent selected from the group consisting of placental derived extracellular matrix, placental derived cell conditioned media, placental exosomes, placental perfusate, wherein the composition with additional constituents delivered via microinfusion are included.

Methods for treating lines, wrinkles, elastosis, extracellular matrix depletion, fibroblast depletions, photo damage, acne scars, hair loss with the disclosed compositions are provided as well. A dermal rejuvenation composition of the instant disclosure is intended for various applications including delivery as 1) dermal fillers, subcutaneous delivery, deep dermis applications. 2) in combination with a cosmetic formulation of Botulin toxin (BOTOX, Allergan Aesthetics) for superficial skin lines and sagginess 3) skin care as peels, masks and microdermabrasion to epidermis, 4) skin composition for epidermis/dermis. 5) Hair follicle regeneration and hair restoration

A dermal rejuvenation composition of the instant disclosure, which comprises, inter alia, placenta derived biomaterials addresses clinical unmet conditions such as photodamaged skin, anti-aging, acne, allergic and inflammatory skin conditions such as rosacea, solar elastosis and hair follicle restoration and regeneration.

The key benefits of a dermal rejuvenation composition as disclosed herein include improved skin elasticity, reduction in fine lines and wrinkles, brighter skin, improved hyperpigmentation, improvement in skin smoothness by reduced appearance of pores, enhanced hydration, and increased moisture and barrier function, and skin plumpness, to name only a few.

### Example 1. Development of a Dermal Rejuvenation Composition of the Instant Disclosure CYNK-201 Phenotype & Characterization

The base composition ingredients include but are not limited to the following: Propanediol, Glycerin, Water, Cyanocobalamin, Sodium Hyaluronate, Sodium PCA, Sodium Lactate, Arginine, Aspartic Acid, PCA, Glycine, Alanine, Serine, Valine, Proline, Threonine, Isoleucine, Histidine, Phenylalanine, Ascorbic Acid, Inositol, Thiamine HCl, Niacinamide, Sodium Riboflavin Phosphate, Pyridoxine HCl. The composition is stable at room temperature and has a shelf life of over 2 years. Additionally, to provide broader benefits, another ingredient that can be included is Sodium Retinoyl Hyaluronate.

These base compositions are supplemented with the additional constituents selected from the group consisting of placental-derived ingredients, placental adherent mesenchymal-like cells, (PDAC) conditioned media, PDAC-derived micronutrients, placental exosomes, placental perfusate and placental extracellular matrix (ECM) to provide personalized solutions for different skin conditions, including skin elastosis, hyperpigmentation, skin aging, hair restoration and maintenance. A dermal rejuvenation composition as described herein proactively targets hard-to-address, specific skin needs by filling the dermis of the skin. The composition final pH was around 5.5 to about 6.0.

### Example 2. Clinical Safety Study: Repeated Insult Patch Test

The objective of this study was to determine the potential of a test material, Dermal Rejuvenation Composition, to elicit dermal irritation and/or induce sensitization following repeated patch applications.

An established, standardized procedure for clinical testing designed to ensure the well-being of clinical study subjects and the generation of reliable study data was followed. The study was conducted in accordance with the study protocol Standard Operating Procedures. In addition, the study was conducted following applicable ICH GCP standards to ensure reliability of data, subject safety and confidentiality.

A total of 57 male and female subjects, ranging in age from 20 to 70 years who met all of the inclusion criteria and none of the exclusion criteria as outlined in the study protocol, were selected for study participation.

The Institutional Review Board (IRB) reviewed and approved the clinical study protocol, investigational test product formulas, and Informed Consent Form prior to study initiation in accordance with Title 21 of the Code of Federal Regulations (CFR), Parts 50 and 56. Additional study documentation may have been submitted to the IRB for review and approval at the discretion of the Principal Investigator or at the request of the IRB.

Each subject was given a copy of the IRB-approved Informed Consent Form (ICF) had the nature and the purpose of the study explained to them by CRL personnel. Prior to entry into the study, the subject gave voluntary written consent to participate by signing the ICF. The Principal Investigator retains the original signed Informed Consent Form in the subject's file and gave a copy of the Informed Consent Form to the subject.

Study Evaluations

| The following Dermal | |
|---|---|
| Scoring System was used: | |
| Dermal Score | Description |
| 0 | No visible skin reaction |
| + | Barely perceptible erythema |
| 1+ | Mild erythema |
| 2+ | Well defined erythema |
| 3+ | Severe erythema and edema |
| 4+ | Erythema and edema with vesiculation |

Letter Codes

| | | | |
|---|---|---|---|
| E = Edema | S = Spreading of reaction | D = Oozing, crusting, | F = Follicular irritation |
| | beyond patch site | and/or superficial erosions | with or without pustule |
| | | | formation (folliculitis) |
| P = Peeling | V = Vesiculation | d = Dryness/scaling | SD = Site Discontinued |
| Pa = Papules | B = Burning | Ho = Hypopigmentation | ST = Site Terminated |
| I = Itching | Sc = Scabbing | Hr = Hyperpigmentation | NP = No patching |
| X = Subject Absent | Ex = Excoriation | C = Changed Site | — = No reading |

### Test Method

This study was conducted according to clinical study protocol CL1.0 IRB 2021

### Study Results

Completed and Discontinued Subjects

| A total of 55 subjects | |
|---|---|
| completed the study. | |
| Discontinued subjects are listed | |
| below: Subject Number | Reason for Discontinuation |
| 74 | Lost to follow up |
| 82 | Lost to follow up |

### Dermal Evaluations

Individual dermal scores recorded during the Induction and Challenge Phases appear in Table I for subjects that elicited dermal reactions and/or were discontinued. All other subjects did not exhibit any dermal reactions throughout the course of the entire study and had scores of ‘0’.

### Conclusion

Based on the test population of 55 subjects and under the conditions of this study, the test material identified as RD2302A Dermal Rejuvenation Composition did not demonstrate a clinically significant potential for eliciting dermal irritation or inducing sensitization.

TABLE 2
Summary of Dermal Scores

| Subject | Induction Scores | | | | | | | | | Challenge Scores | | | |
|---|---|---|---|---|---|---|---|---|---|---|---|---|---|
| Number | 1 | 2 | 3 | 4 | 5 | 6 | 7 | 8 | 9 | 24 Hr. | 48 Hr. | 72 Hr. | 96 Hr. |
| 74 | DISCONTINUED | | | | | | | | | | | | |
| 82 | 0 | DISCONTINUED | | | | | | | | | | | |
| 87 | 0 | 0 | 0 | 0 | 0 | 0 | 0 | 0 | 0 | 0 | 0 | 0 | X |
| 107 | 0 | 0 | + | + | 0 | 0 | 0 | 0 | 0 | 0 | 0 | 0 | 0 |

*All other 53 subjects except the above listed did not exhibit any dermal reactions throughout the course of the entire study and had scores of '0' .*

APPENDIX I
Subject Demographics

| Subject | | |
|---|---|---|
| Number | Age | Sex |
| 61 | 33 | F |
| 62 | 63 | M |
| 63 | 70 | F |
| 64 | 62 | M |
| 65 | 67 | F |
| 66 | 35 | F |
| 67 | 66 | F |
| 68 | 68 | F |
| 69 | 60 | F |
| 70 | 52 | M |
| 71 | 48 | F |
| 72 | 70 | F |
| 73 | 56 | F |
| 74 | 47 | F |
| 75 | 37 | M |
| 76 | 25 | F |
| 77 | 25 | M |
| 78 | 59 | F |
| 79 | 65 | M |
| 80 | 65 | M |
| 81 | 59 | F |
| 82 | 37 | M |
| 83 | 65 | F |
| 84 | 61 | F |
| 85 | 20 | M |
| 86 | 58 | F |
| 87 | 55 | M |
| 88 | 54 | F |
| 89 | 57 | F |
| 90 | 58 | F |
| 91 | 65 | F |
| 92 | 30 | M |
| 93 | 59 | F |
| 94 | 61 | F |
| 95 | 65 | F |
| 96 | 51 | M |
| 97 | 38 | M |
| 98 | 23 | F |
| 99 | 65 | M |
| 100 | 57 | F |
| 101 | 48 | F |
| 102 | 43 | F |
| 103 | 28 | F |
| 104 | 26 | F |
| 105 | 56 | F |
| 106 | 55 | F |
| 107 | 53 | F |
| 108 | 40 | F |
| 109 | 23 | M |
| 110 | 56 | F |
| 111 | 51 | F |
| 112 | 41 | F |
| 113 | 53 | F |
| 114 | 62 | F |
| 115 | 35 | F |
| 116 | 54 | F |
| 117 | 53 | M |

### Example 3. Clinical Safety Study RD4341A Repeated Insult Patch

### Objective

The objective of this study was to determine the potential of a test material to elicit dermal irritation and/or induce sensitization following repeated patch applications.

### Study Results

### Completed and Discontinued Subjects

A total of 59 subjects completed the study. The study was discontinued for subject #36, who was lost to follow up.

### Dermal Evaluations

Individual dermal scores recorded during the Induction and Challenge Phases appear in Table I for subjects that elicited dermal reactions, missed a visit, and/or were discontinued.

All other subjects did not exhibit any dermal reactions throughout the course of the entire study and had scores of ‘0’.

### Conclusion

Based on the test population of 59 subjects and under the conditions of this study, the test material identified as RD2321A did not demonstrate a potential for eliciting dermal irritation or inducing sensitization.

TABLE 3
Summary of Dermal Scores

| Subject | Induction Scores | | | | | | | | | Challenge Scores | | | |
|---|---|---|---|---|---|---|---|---|---|---|---|---|---|
| Number | 1 | 2 | 3 | 4 | 5 | 6 | 7 | 8 | 9 | 24 Hr. | 48 Hr. | 72 Hr. | 96 Hr. |
| 36 | DISCONTINUED | | | | | | | | | | | | |
| 49 | 0 | 0 | 0 | 0 | 0 | 0 | 0 | 0 | 0 | 0 | X | 0 | 0 |
| 59 | 0 | 0 | 0 | 0 | 0 | 0 | 0 | 0 | 0 | 0 | 0 | X | 0 |

*All other 57 subjects except the above listed did not exhibit any dermal reactions throughout the course of the entire study and had scores of '0' .*

APPENDIX I
of this Study-Subject Demographics

| Subject | | |
|---|---|---|
| Number | Age | Sex |
| 01 | 61 | F |
| 02 | 61 | F |
| 03 | 65 | M |
| 04 | 55 | F |
| 05 | 47 | F |
| 06 | 66 | F |
| 07 | 69 | M |
| 08 | 63 | F |
| 09 | 59 | F |
| 10 | 20 | F |
| 11 | 59 | F |
| 12 | 57 | M |
| 13 | 52 | F |
| 14 | 48 | M |
| 15 | 54 | F |
| 16 | 30 | F |
| 17 | 38 | M |
| 18 | 53 | M |
| 19 | 63 | M |
| 20 | 33 | F |
| 21 | 64 | F |
| 22 | 48 | F |
| 23 | 32 | M |
| 24 | 51 | M |
| 25 | 56 | F |
| 26 | 66 | M |
| 27 | 67 | F |
| 28 | 69 | M |
| 29 | 67 | F |
| 30 | 46 | F |
| 31 | 19 | M |
| 32 | 40 | F |
| 33 | 51 | F |
| 34 | 26 | F |
| 35 | 62 | M |
| 36 | 40 | M |
| 37 | 32 | F |
| 38 | 54 | F |
| 39 | 54 | F |
| 40 | 57 | F |
| 41 | 54 | M |
| 42 | 57 | F |
| 43 | 44 | F |
| 44 | 46 | F |
| 45 | 58 | F |
| 46 | 55 | M |
| 47 | 56 | F |
| 48 | 68 | F |
| 49 | 54 | M |
| 50 | 41 | F |
| 51 | 64 | F |
| 52 | 68 | F |
| 53 | 62 | M |
| 54 | 58 | F |
| 55 | 20 | F |
| 56 | 61 | F |
| 57 | 40 | F |
| 58 | 64 | F |
| 59 | 30 | F |
| 60 | 66 | F |

### Example 4. Composition Microbial Testing and Shelf-Life Stability

An example is provided of microbial challenge testing and determining shelf-life stability. The temperature that the tests were performed included ambient and accelerated temperature.

Description: Skin Rejuvenation Composition Sample ID: RD2321A. Lot Number RD2321A.

| | | | |
|---|---|---|---|
| Total Aerobic Microbial Count | <10 | CFU/g | USP <2021> |
| E.coli | Absent | per 10 grams | USP <2022> |
| Staphylococcus aureus | Absent | per 10 grams | USP <2022> |
| Salmonella | Absent | per 10 grams | USP <2022> |
| Total Yeast & Mold | <10 | CFU/g | USP <2021> |
| Yeast * | <10 | CFU/g | USP <2021> |
| Mold * | <10 | CFU/g | USP <2021> |
| Pseudomonas aeruginosa | Absent | per 10 grams | USP <62> |

The test results demonstrate the absence of microbial content in all dermal rejuvenation compositions of the instant disclosure at all tested times.

### Example 4: Isolation and Purification of pExo from Human Postpartum Placenta

Full-term human placentas procured through donor with full consent are processed to small tissue fragments with dissection and placed in sterile cell culture containers such as roller bottle bioreactors filled with sterile saline solution. The placenta tissues are incubated at room-temperature or defined cultured conditions such as incubators for at least 2 hours and then the supernatant of the tissue-culture is isolated with centrifugation. The supernatants of the isolation are then centrifuged with two step centrifugations: first step as 3000 g and second step of 10,000 g to obtain a supernatant of pExo for ultra-centrifugation. The supernatant from the 10,000 g centrifuge (S10,000 g) is then used for ultra-centrifugation at 100,000 in which the supernatant is discarded and the pellet contained. The pellet is then resuspended in solutions such as PBS, saline, and used as constituent of the Dermal Rejuvenation Composition of the instant disclosure. Protein concentration of pExo is quantified with BCA protein assay method. The size is determined by NanoSight. The cytokine composition of pExo is measured with MILLIPEX-MAP human cytokine/chemokine-PX41.

### Example 5. Placental Adherent Mesenchymal-Like Cell PDAC Population Derived Supernatants and Micronutrients

Placental derived PDAC are isolated from normal, full-term human placental tissue. Cells are culture expanded as a plastic-adherent undifferentiated in vitro population. PDAC are ≥95% CD34−, CD10+, CD105+ and ≥84% of the cells as CD200+. The remaining cells are part of the same continuous population but are not considered positive by flow cytometry because their signal is too weak. Cells are roughly spherical in mono-dispersed suspension with an average cell diameter of 19+/−1 μm.

The cells have the genetic make-up of the baby, not the mother. When cells are cultured for over 24 hours, cell micronutrients are collected and used as one of the constituents of the Dermal Rejuvenation Composition. Physical embodiments of the cell micronutrients include, but are not limited to, liquid or solid, frozen, lyophilized or dried into a powder. Cell micronutrients contain many of the original components of the medium, as well as a variety of cellular metabolites and secreted proteins, including, for example, biologically active growth factors, anti-inflammatory mediators and secreted extracellular matrix proteins. The micronutrients are used as one of the constituents of the dermal rejuvenation composition.

### Example 7. Evaluation of Topical Application of a Dermal Rejuvenation Composition of the Instant Disclosure in Healthy Volunteers

The ingredients were directly micro-deposited into the superficial dermis of the skin (with minimal downtime and maximum results. 3 cc of the Dermal Rejuvenation Composition was used for the treatment. A 3 cc of the composition was used and a micro-injecting device, consisting of 200-600 micron depth micro needles was used. Additional types of devices can be used including rollers or microinjectors. A topical application without micro infusion also provides benefits to the skin. The dermal rejuvenation Composition was applied around the entire face area using fingertips and patted in gently until thoroughly absorbed. Following treatment, the following benefits were demonstrated as early as 24 hours post treatment: Improved skin elasticity, Reduced appearance of fine lines and wrinkles around eyes and mouth, brighter skin, Improved hyperpigmentation, Smoother skin, enhanced hydration, skin Plumpness, and evenness. These benefits were evaluated on a scale 1-5 as subjective scores.

### Example 8. Generation of Extracellular Matrix from Decellularized Placental Chorion

As one of the constituents of Dermal Rejuvenation Composition, placental extracellular matrix is utilized in the composition. Examples include but are not limited to decellularized chorionic plate, amnion, umbilical cord, umbilical cord plasma or serum, or a combination of the above.

The chorionic plate was mechanically dissected from the placenta. Following dissection, the plate was mechanically scraped to remove the residual placental tissue. The chorionic plate was then placed in a bottle with 2M NaCl solution and incubated in an orbital shaker for greater than 4 hours. The solution was then changed to sterile water and incubated in an orbital shaker for up to 24 hours. The exposure of the chorionic plate to a high concentration of NaCl, followed by water constitutes an “osmotic shock” to the tissue. The osmotic shock serves to lyse cells, remove blood, blood components, cells, and cell debris from the tissue. To remove embedded cells (e.g., fetal trophoblasts or epithelial cells), the tissue was rinsed overnight in 2% deoxycholic acid. The deoxycholic acid was then removed from the tissue with several sterile water washes. At this point of manufacturing, the in-process product is composed of a densely packed, highly organized native extra-cellular matrix. The wet tissue is then homogenized in a commercial blender and then lyophilized by first freezing the blended chorionic plate at −80° C. until the tissue is frozen and freeze-drying in a lyophilizer for 16-18 hours. The lyophilized placental matrix, was then cryomilled using a Cryomill into desired particulate size. The extracellular matrix could be used as one of the constituents of the dermal rejuvenation composition.

### Example 9. Generation of Decellularized Placental ECM

As one of the constituents of a Dermal Rejuvenation Composition of the instant disclosure, placental extracellular matrix will be used. Another example incudes decellularized placental extracellular matrix.

Processing/decellularization of the placenta is conducted following ASTM-F3354 standard guide [8], using aseptic technique in a Class 100 BSC (biological safety cabinet) in an ISO 7 cleanroom, using sterile, single-use consumables. The process begins with the procurement of placenta, obtained from a normal, full-term delivery.

The ECM component is sourced from whole human placentas, from which the umbilical cord is removed. The frozen placenta is released from quarantine for processing and is thawed at 4-8° C. for 72-96 hours and transferred to a BSC. Each placenta is processed as a separate lot. The placenta is cleaned to remove excess blood and blood clots and cut into small 0.5×0.5-inch segments. The cut placental segments are suspended in sterile water and are homogenized using an Omni mixer homogenizer (with a 2-inch rotor knife probe). The homogenization step serves to generate small tissue particulates, with increased surface area, allowing for more effective separation and removal of cells and cellular debris from the placental tissue. The homogenized tissue from a single placenta is transferred into a sterile processing container of 2M sodium chloride (NaCl) solution. The tissue is washed repeatedly with sterile 2M NaCl by shaking on an orbital shaker; the NaCl solution is exchanged by centrifugation to settle tissue particulate, followed by removal of the supernatant, and refreshed with additional sterile 2M NaCl solution. The placental tissue is incubated at controlled room temperature (20 to 25° C.) for up to 24 hours while shaking in sterile 2M NaCl solution. The supernatant is removed from the sterile processing container after the completion of the water wash and replaced with a solution of 2 mM magnesium chloride (MgCl2) and 10 units/L of Benzonase® and is incubated up to 24 hours on an orbital shaker at room temperature to aid in degradation of all forms of nucleic acids (deoxyribonucleic acid (DNA) and ribonucleic acid (RNA). Benzonase is an endonuclease that degrades all forms of nucleic acids (DNA & RNA) into shorter polynucleotide fragments that are more readily removed by subsequent sterile water washes.

Following removal of Benzonase, the tissue is subjected to detergent washes. Upon completion of the detergent washes, the tissue is washed with sterile water. The decellularized placental ECM is then transferred into a sterile disposable tray, frozen at −80° C., lyophilized and stored at room temperature in an enclosed container post lyophilization. The lyophilized placental ECM wafer is further processed by cryomilling to a finely powdered form-placental ECM (pECM). The placental ECM can be used as one of the constituents of the dermal rejuvenation composition.

### Example 10. A Pilot Clinical Evaluation of Dermal Rejuvenation Composition Containing Decellularized Micronized Placental ECM on the Appearance of Hands Skin Surface Evenness, Dark Spots and Fine Lines and Wrinkles

The placental extracellular matrix suspended in base composition was directly micro-deposited into the superficial dermis of the hands. 3 cc of the Dermal Rejuvenation Composition was used for the treatment combined with placental ECM. The most effective results come when the Dermal Rejuvenation Composition was deposited through the skin via a microneedling device. A 3 cc of the composition was used and a micro-injecting device consisting of 200-600 micron depth micro needles. Additional types of devices can be used including rollers or microinjectors. A topical application without micro infusion also provides maintenance results and benefits to the skin. The Skin Rejuvenation Composition was applied around the entire hands area without a device and patted in gently until thoroughly absorbed. Following treatment, the following benefits were demonstrated as early as 24 hours post treatment: Improved skin elasticity, reduced appearance of fine lines and wrinkles, brighter skin, improved hyperpigmentation, smoother skin, enhanced hydration, plumpness, and evenness. These benefits were evaluated on a scale 1-5 as subjective scores.

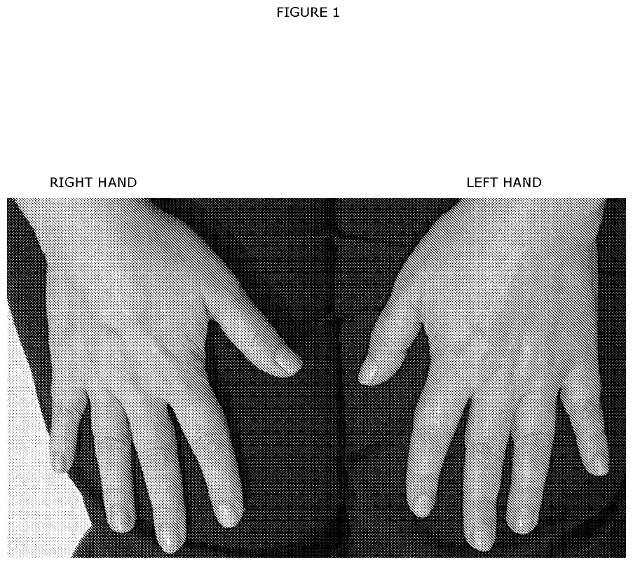

In FIG. 1 from this evaluation, a subject's hands were photographed following application of placental extracellular matrix treatment. The subject's left hand was treated with a dermal rejuvenation composition of the instant disclosure, while the subject's right hand was treated with a saline solution.

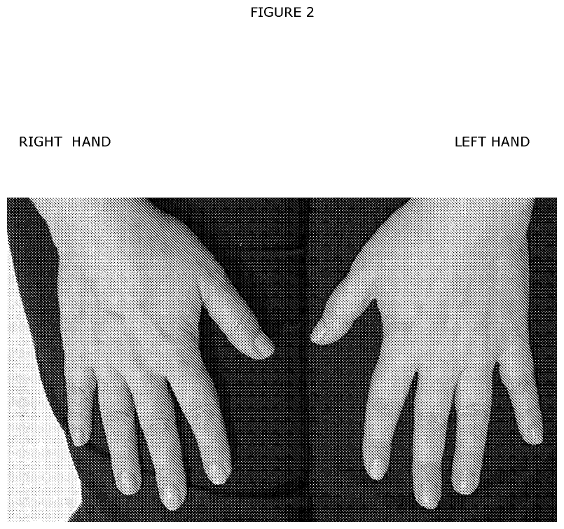

In FIG. 2 of this evaluation, a subject's hands were photographed after five minutes of exposure to a dermal rejuvenation composition of the instant disclosure. The subject's left hand was treated with a dermal rejuvenation composition of the instant disclosure, while the subject's right hand was treated with a saline solution.

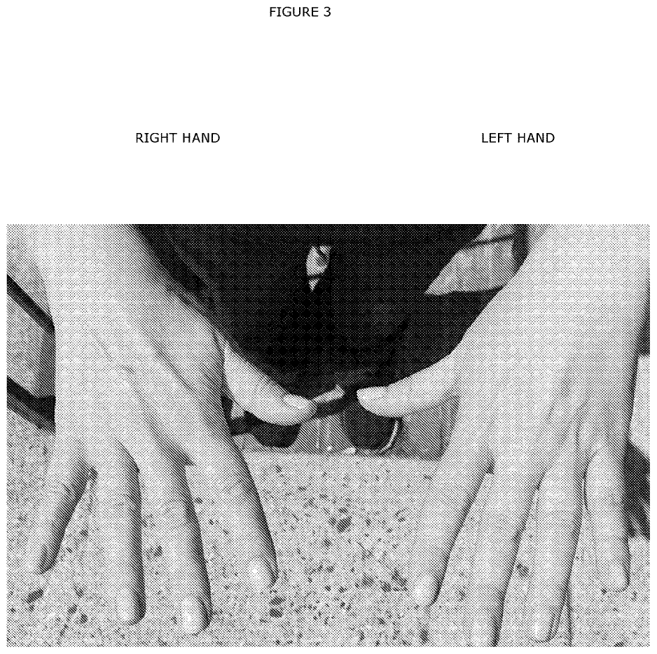

In FIG. 3 of this evaluation, a subject's hands were photographed following administration of a dermal rejuvenation composition of the instant disclosure. The subject's left hand was treated with a dermal rejuvenation composition of the instant disclosure and the subject's right hand was treated with a saline solution. After five minutes of exposure, the treatments were wiped off gently. The right-hand skin showed the appearance of smoother and plumper skin as compared to the skin of the left hand.

### Example 11. Dermal Rejuvenation Composition Calming Effects

In another example, to minimize the inflammatory response post aesthetic procedure including, but not limited to radiofrequency micro-needling technology for deep tissue remodeling, skin laser ablation, dermal peeling or skin resurfacing, other constituents might be included from a group consisting of D-Panthenol, Rosemary Extract, Sodium Lactate, Centella Asiatica Extract, Ceramides, Tocopherol Acetate. A skin mask containing a dermal rejuvenation composition of the instant disclosure is included. The final pH of the composition was around 5.5-6.0 Viscosity: Spindle-TD, S94; Speed-2. rpm; Range-3,000-5,000 mPas.

### Example 12. Different Forms of a Dermal Rejuvenation Composition of the Instant Disclosure

Different forms will be generated, including most concentrated for office procedures and less concentrated for consumer use. Different constituents' concentration will be used from the group consisting of placental-derived ingredients, placental adherent mesenchymal-like cells, conditioned media, placental cell-derived micronutrients, placental exosomes, placental perfusate, umbilical cord blood plasma or serum, and placental extracellular matrix (ECM) to provide personalized solutions for different skin conditions, including skin elastosis, hyperpigmentation, skin aging, dark circles, eye puffiness, hair follicle regeneration. The dermal rejuvenation composition proactively targets these different skin needs.

### REFERENCES

1. Clin Cosmet Investig Dermatol v.8; 2015
2. Brigido S A, Carrington S C, Protzman N M. The Use of Decellularized Human Placenta in Full-Thickness Wound Repair and Periarticular Soft Tissue Reconstruction: An Update on Regenerative Healing. Clin Podiatr Med Surg. 2018 January; 35(1):95-104. doi: 10.1016/j.cpm.2017.08.010. Epub 2017 Oct. 23. PMID: 29156171.
3. Bhatia, M B, et al. “A Human Placenta-Derived Decellularized Connective Tissue Matrix Supports Cellular Functions Involved in Wound Healing Processes.” SAWC Poster
4. Smiell J M, Treadwell T, Hahn H D, Hermans M H. Real-world Experience With a Decellularized Dehydrated Human Amniotic Membrane Allograft. Wounds. 2015 June; 27(6):158-69. PMID: 26061491.
5. Timothy J. Keane, Ricardo Londono, Neill J. Turner, and Stephen F. Badylak. “Consequences of ineffective decellularization of biologic scaffolds on the host response,” Biomaterials 2012:33:1771-1781.
6. Gilbert T W, Freund J, and Badylak S F. Quantification of DNA in Biologic Scaffold Materials. J Surg Res 2009; 152(1): 135-139.
7. ASTM-F3354-19 Standard Guide for Evaluating Extracellular Matrix Decellularization Processes.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A dermal rejuvenation composition comprising:
(a) a placenta derived biomaterial;
(b) hyaluronic acid or an analog of derivative thereof, or a salt thereof;
(c) a vitamin;
(d) an amino acid
(e) a microelement; and
(f) a lipid compound.

2. The dermal rejuvenation composition of claim 1, wherein the placenta derived biomaterial comprises:
(a) placenta derived extracellular matrix;
(b) placenta derived cell conditioned media;
(c) placenta derived exosomes;
(d) placenta derived perfusate; or
(e) any combination of (a)-(d).

3. The dermal rejuvenation composition of either of claim 1 or 2, wherein the hyaluronic acid, an acid or derivative thereof, or a salt thereof comprises (a) a high molecular weight hyaluronan, (b) a fluid mixture of hylan A and hylan B polymers, (c) sodium hyaluronate, (d) a highly purified, partially cross-linked sodium hyaluronate, (e) a linear polysaccharide with disaccharide repeats of d-glucuronic acid and N-acetyl-d-glucosamine, (f) a high molecular weight viscoelastic linear polysaccharide and disaccharide repeats of d-glucuronic acid and n-acetyl-d-glucosamine, (g) esters of hyaluronic acid, or (h) any combination of (a)-(g).

4. The dermal rejuvenation composition of any of claims 1-4, wherein the vitamin comprises: (a) retinol (vitamin A); (b) ascorbic acid as well as analogs and derivatives thereof, e.g., ascorbyl palmitate (vitamin C); (d) sodium riboflavin phosphate, thiamine HCl; (e) niacin or an niacin amide (vitamin B3); (f) pantothenic acid (vitamin B5); (g) pyridoxine (vitamin B6); (h) biotin (vitamin H); (i) phylloquinone (vitamin K1); (j) menaquinone (vitamin K2); (k) calciferol (vitamin D); (l) alpha-linolenic acid (ALA) and linoleic acid (LA) (vitamin F); (m) coenzyme Q10 (vitamin Q10); (n) Cyanocobalamin (vitamin B12); or any combination of (a)-(n).

5. The dermal rejuvenation composition of any of claims 1-4, wherein the amino acid comprises: (a) Arg; (b) Asp; (c) Ala; (d) Ser; (e) Val; (f) Pro; (g) Thr; (h) Ile; (i) His; (j) His; (k) Phe; or (l) any combination of (a)-(k).

6. The dermal rejuvenation composition of any of claims 1-5, wherein the lipid compound comprises: (a) glycerin; (b) nourishing oils; or (c) a combination of (a) and (b).

7. The dermal rejuvenation composition of any of claims 1-6, further comprising a carbohydrate.

8. The dermal rejuvenation composition of claim 7, wherein the carbohydrate comprises inositol, a monosaccharide, or glycerin.

9. The dermal rejuvenation composition of any of claims 1-8, further comprising retinol or a derivative thereof.

10. The dermal rejuvenation composition of claim 9, wherein the retinol or a derivative thereof is sodium retinoyl hyaluronate.

11. The dermal rejuvenation composition of any of claims 1-10, wherein the composition is aqueous.

12. The dermal rejuvenation composition of claim 11, having a pH of about 5.0 to about 6.5.

13. The dermal rejuvenation composition of either of claim 11 or 12, having a pH of about 5.5 to about 6.0.

14. A method for treating a dermal disease or condition in a subject, comprising administering a therapeutically effective amount of the dermal rejuvenation composition of any of claims 1-13 to the subject.

15. The method of claim 15, wherein the dermal disease or condition is selected from the group consisting of: (a) wrinkle lines; (b) wrinkles; (c) elastosis; (d) (extracellular matrix depletion, (e) fibroblast depletions; (f) photodamage; (g) a scar; and (h) any combination of (a)-(g).

17. The method of claim 16, wherein the scar comprises: (a) an acne scar; (b) a burn scar; (c) a laceration scar; or (d) any combination of (a)-(c).

18. The method of any of claims 14-17, wherein the therapeutically effective amount of the dermal rejuvenation composition is administered to the subject topically, intradermally, or subcutaneously.

19. The use of the dermal rejuvenation composition of any of claims 1-13 for use in a method for treating a dermal disease or condition in a subject.

20. The use of claim 19, wherein the dermal disease or condition is selected from the group consisting of: (a) wrinkle lines; (b) wrinkles; (c) elastosis; (d) extracellular matrix depletion; (e) fibroblast depletions; (f) photo damage; (g) a scar; and (h) any combination of (a)-(g).

21. A kit comprising
(i) a dermal rejuvenation composition of any of claims 11, 12 or 13,
(ii) placenta derived extracellular matrix in (a) a liquid form, or (b) in a powder form that can reconstituted into a liquid form prior to administration; and
(iii) an applicator.
